(19) United States
(12) Patent Application Publication
(10) Pub. No.: US 2013/0296784 A1
(43) Pub. Date: Nov. 7, 2013

(54) PUMP AND PREFILLED BAG/ANAESTHESIA - ANALGESIA SET

(71) Applicant: MICREL MEDICAL DEVICES S.A., Gerakas (GR)

(72) Inventor: Achilleas Tsoukalis, Gerakas (GR)

(73) Assignee: MICREL Medical Devices S.A., Gerakas (GR)

(21) Appl. No.: 13/886,682

(22) Filed: May 3, 2013

(30) Foreign Application Priority Data
May 4, 2012 (GR) 20120100242

(51) Int. Cl. A61M 5/142; A61J 1/10; A61M 5/50

(52) U.S. Cl. CPC A61M 5/14212; A61M 5/5086; A61J 1/10
USPC 604/111; 604/151

(57) ABSTRACT

The invention is directed to an analgesia and/or an anesthesia drug/device, system and an analgesic and/or an anaesthetic method utilizing a drug storage bag prefilled pharmaceutically or in a pharmaceutical compounding facility with a drug which system or method involves long-term stability of the thug prefilled storage bag, thus enabling the simplification of both, home treatment and hospital treatment procedures, in particular, the system or method is utilizing a storage bag comprising multi-layered flint. the surface of which is drug-compatible while subsequent layers constitute a barrier against any gases, e.g. oxygen, carbon dioxide, water vapour and/or humidity, and optionally ultraviolet radiation. This system and method comprises preferably a label/marking, providing information in a visual and/or wireless way to the pump system and the supporting services, directly or through the internet, in order to avoid medical errors regarding the administration of analgesic and/or anesthetics drugs. The system and method also enables for the unlocking of any drug prefilled storage bag, preferably a drug cassette, which is protected against abuse.

The present invention pertains to a system and a method for administering an anaesthetic and/or an analgesic drug to a patient comprising a pump for administering an anaesthetic and/or an analgesic drug preferably via the IV (intravenous) route or the central nerve block (spinal, epidural anaesthesia) or the peripheral nerve Nock and a storage bag containing the anaesthetic and/or the analgesic drug.

In the state of the art, a method for preparing and packaging a drug is provided as we as a method for the continuous and safe administration of same to patients. For example reference is made to U82012/0016295 or to its European counterpart EP 2 410 448 A2.

The most widespread pharmaceutically used substances in analgesia and/or in anaesthesia are generic, like morphine in all its forms (morphine sulphate, morphine hydrochloride), or oxycodone or ropivacaine, and henceforth are very low in price.

The packaging for long-term storage of such substances in analgesia and/or in anaesthesia for many years was glass, wherein glass offers protection against volatility and absorbency. During the last decade the production of multilayer sheets of plastic matter allowed the long-term plastics-packaging of drugs, i.e., for 2 or more years.

The drug storage bags of the infusion pump sets in the state of the art are made of a single layer flexible PVC sheet or a polypropylene sheet (named “Non-PVC”) and may not be prefilled for long-term storage. Otherwise an alteration in drug concentration will occur, water-vapour exiting through the plastic and oxygen plus other oxide anion free radicals oxidants entering as time progresses, i.e. such drug storage bags exhibit poor long-term use and poor drug stability.

In the case of infusion with a portable pump, either a disposable infusion set is used bearing the pump manufacturer's spike plus some pharmaceutical company's drug prefilled bag which is the same for all pumps, portable or not, or during the treatment-application procedure to fill locally the bag supplied by the company supplying the pump, e.g. as in case of a Rythmic™ Full set of the Rythmic™ portable peristaltic pump offered by Micrel Medical Devices for direct or short-term use.

Prefilling for long-term storage of such an adapted to the pump bag, has size-associated advantages on account of the bag being manufactured in a shape and size adapted to the pump, this being an important advantage in portable use, it is common practice today for the commercial (made by the pharmaceutical companies) bags to be placed in banana- or shoulder-type pouches equipped with pump, in more than double the required size.

Another advantage is easy placement into the pump on account of the infusion set plus the drug comprising a single module, which is easily installed in place either in the pump cavity or as a cassette.

The infusion can be carried out in really portable conditions, in any pump-bag direction, if the pump bag is made of a material bearing micro-striations internally, in order to avoid the bag's walls collapsing and sticking together when the pump is in suction mode. When collapsing and sticking together of the walls occurs, bag emptying is prevented ending-up in under-infusion, which is not observed when the bag is vented and hanged vertically, e.g, by placing the hag on a pole, i.e., not in portable use. Prefilled bags are all supplied with a smooth internal surface since they are mainly used on poles connected to bedside pumps.

The localised filling of the bag (usually made of a single layer PVC film or polypropylene film) in the Hospital, at home or the small-quantity pharmaceutical compounding facility, bears the cost of syringes, gloves etc., plus the risks of nurse or formulator piercing. The stability duration of such filling in simple plastic sheets is only a few days, depending on the drug and the material, and a strategy for cost and risk reduction cannot be found. All of these risks should he avoided as will be reported analytically below in the context of the present invention.

In the practice of hospital treatment in-use errors are observed, wherein the wrong drug or drug concentration is infused, to the wrong patient, or the wrong protocol is followed, leading to patient apnoea and death. These risks should be avoided and the problem resolved by the present invention.

The high cost of analgesia for companies offering treatment at home, due to the fact that they have to carry the recently filled bag, which for compounding in PVC has a stability of is only one to two weeks. Conversely, a high stability prefilled bag fitted with corresponding safety labelling/marking would allow bag collection from the local drugstore, with the respective nurse-cost saving.

It is the intention of the present invention to provide a pump system, fitted with a prefilled analgesia and/or anaesthesia bag, resolving all the above problems and affording the advantages in an easy and effective way.

### SUMMARY OF THE INVENTION

The invention is directed to an analgesia and/or to an anaesthesia drug/device system and an analgesic method and/or anaesthetic method utilizing a drug storage bag prefilled pharmaceutically or in a pharmaceutical compounding facility with a drug which system or method involves long-term stability of the drug prefilled storage bag, thus enabling the simplification of both, home treatment and hospital treatment procedures. In particular, the system or method is utilizing, a storage bag comprising multi-layer films, drug-compatible, -with inner layers to constitute a barrier against any gases e.g. oxygen, carbon dioxide, water vapour and/or humidity, and optionally ultraviolet radiation. This to system and method comprises preferably a label/marking, providing information in a visual and/or wireless way to the pump system and the supporting services, directly or through the internet, in order to avoid medical errors regarding the administration of analgesic drugs. The system and method also enables for the unlocking of any drug prefilled storage bag, preferably a drug cassette, which is protected against abuse.

The invention preferably concerns an analgesics and/or an anaesthetics infusion pump combined with a drug-prefilled bag/set, wherein said term ‘prefilled’ has the meaning as commonly used in a pharmaceutical authorization, e.g. the term is meant to define compliance with regulatory requirements, preferably those set forth in the applicable guidelines of the European Medicine Agency (EMA, previously known as EMEA) and more preferably as additionally set forth with the applicable guidelines of the Food & Drug Administration (FDA) in the USA, for a new drug packaging (pharmaceutical envelope) having a longer Shelf-life, particularly longer than one month, following a filling method in pharmaceutical compounding facility designed for the exclusive cooperation between the storage bag and the pump which guarantees greater safety when infusing, in particular dangerous analgesics. Accordingly, the term “pre-filled” means that the bag or bag set is filled with the analgesic and/or the anaesthetic drug by a pharmaceutical company, in such way that it can be shelved and/or supplied by conventional pharmaceutical supply chain management. This means, in contrast to the state of the art, that the bag or bag set is not filled in a hospital, clinic or a pharmacy directly odor to individual administration to a patient, but can be produced in an industrial pharmaceutical manner more efficiently and on beforehand. Accordingly, using a multilayer bag, a compounding method of filling (e.g. by providing a compounder in each country) can offer the desired stability of analgesics and/or anaesthetics (long, time on the shelf) so that storage at Hospital can be profitable avoiding burden and errors of local filling. Thus, the term “long-term” in the context of the invention is meant to designate a longer shelf-life of the drug-prefilled bag/set of at least 1 month, preferably at least 2 months, more preferably at least 3 months, and most preferably more than a year under standard room conditions.

In the present invention, this being novel over the state of the art, the disposable pump set (with or without a cassette) which may include the bag plus the infusion segment part (i.e., the piping alone) and/or the infusion mechanism, is considered after the pharmaceutical solution prefilling process as a drug/medical device combination and despite the fact that there is only drug distribution, it may be charged to the social security funds as a simple device without the drug, due to the low price of the analgesics and/or anaesthetics involved, or as the sum of both, drug plus device, or drug only in case it happens to be expensive.

In medical practice today such a drug/medical device combination is non existent, there exist bags/pump sets for filling locally, or drugs packed in small sacs linked to the pump set through a spike. Ease of use, error avoiding (i.e. wrong concentration) and lower cost, are the advantages offered by the present invention (set/drug on the shelf.

A part of the present invention is when the bag, in the final packaging, is not linked to the pump, it is preferred that in the full set assembly there is a sub-assembly (bag sub-assembly) comprising the bag and a simple connector e.g., luer connectors, fitting into the pump cavity/cassette, while the rest of the infusion set (second sub-assembly) can be 2e reimbursed as usual. This way the sub-assembly parts may be charged (reimbursed) separately according to certain legislation. The label/marking contains information of a certain drug name with a certain date of expiry, concentration and solvent, for a certain pump family manufactured by a specific company. The present invention because of the long-term stability of the drug in the special bag reported below, and hence the lone-term storage, provides the possibility and the advantage for the prefilling to be either carried out by the pharmaceutical company as a regular drug or in a pharmaceutical compounding facility, such that it has the right labelling/marking identifiable by the rump according to a special protocol as mentioned below, plus zero preparation errors as far as quantity and strict quality procedures are concerned, due to the organised production management system and not occasionaly on patient therapy demand.

### DETAILED DESCRIPTION OF THE INVENTION

The present invention pertains to a drug/medical device system for administering an analgesic drug and/or an anaesthetic drug to a patient preferably through the IV route or the central nerve block or the peripheral nerve block comprising:

(a) a pump for administering at least one analgesic drug and/or anaesthetic drug,

(b) a storage bag or a storage hag prefilled with the at least one analgesic drug and/or anaesthetic drug, and

(c) at least one means whereby the pump to and the storage bag or storage bag set (b) are adjusted to exclusively cooperate with each other when administered to an individual patient,

and wherein the method used to provide the pre-filled storage bag or a storage bag set (b) is in compliance with the guidelines of a competent drug regulatory authority applicable for pre-filled systems, preferably for pre-filled systems for long-term storage.

In the present invention, the drug bag customized for a specific pump or pump group is preferably made of multilayer plastic film, more preferably comprising (or consisting of) an outer layer made of EVA (Ethylene vinyl acetate) and/or polyolefins (preferably polypropylene, polyethylene) in contact with the drug plus Preferably further inner layers made of ethylene-Vinyl alcohol EVOH barrier or AlOx (alumina oxide)—Al2O3, or SiOx, PCTFE (plolychloro-triethylene) homopolymer, or CTFE (chlorotrifluoroethylene), polyaclyonitrile, or LCP liquid crystal polymers, or PVDC Polyvinylidene chloride, and/or a combination thereof, and/or ultraviolet barriers (UV filters) or radio-opaque packaging. intermediate to the barrier layers there may be flexible plastic, adhesive-tie layers. Furthermore the surface finish of the layer in contact with the drug, preferably is not smooth preferably it bears internal micro-striations or any other embossment or texture that makes the inner surface not completely flat in order to avoid the two bag sheets sticking together during suction, and for complete emptying in portable conditions. Next layers, numbers two to seven, preferably are made of EVOH and/or other materials acting as barriers against evaporation and the ingress of gases or UV radiation, This multilayer film material texture properties are similar to the simple monolayer film material since this multilayer film can be produced by co-extrusion process. A second production phase of the multilayer material in this invention can be the lamination to additional multilayer films.

Accordingly, it is preferred in the present invention that it pertains to a drug/medical device system, wherein the storage bag comprises internal striations, preferably internal micro-striations or any other type of ondulation/bumping that keeps the two layers at small distance to allow liquid to pass through under suction conditions as they do not collapse.

Accordingly, it is preferred in the present invention that it pertains to a drug/medical device system, wherein the storage bag is made of a multi-layer flexible sheetequipped with an additional transport barrier against oxygen, carbon dioxide, water vapour and/or humidity, and ultraviolet radiation.

Accordingly, it is preferred in the present invention that it pertains to a drug/medical device system, wherein the drug storage bag is packaged alternatively inside en aluminum/plastic laminated foil bag and Nitrogen gas Modified Atmosphere Packaging. As well known aluminum/plastic foil used in food packaging has best barrier characteristics with extremely low cost.

it is preferred according to the invention that the drug/medical device system comprises a protecting storage case made of cardboard or expanded polystyrene or other foam is sheets so that the set has a mechanical strength protecting packaging.

Accordingly, it is preferred in the present invention that it pertains to a drug/medical device system, wherein the storage bag is adjusted for portable use and more preferably wherein a single storage bag is equipped with an individual external packaging and/or a multitude of storage bags is equipped with a collective packaging.

Packaging of the set may be done in a hard-walled carton box, or with the protection provided by expanded polystyrene or other foam sheets plus an external simple label/marking according to the regulating environment of the drug-device combination.

Prefilling can be achieved by methods known to the person skilled in the art. For example the prefilling can be achieved in the following two ways:

In the normal pharmaceutical procedure, the bag's material is tested for stability (stability test) for every single drug, and its distribution in a prefilled form requires a complete marketing authorisation file, or a file for changes made in packaging, i.e., from material “a” to material “b”, or the same material but in a different size, since drugs are generic supported by a different file. Pharmaceutical prefilling is preferably carried out under vacuum or modified atmosphere in an authorized pharmaceutical company under the supervision of the relevant authorities, observing all the necessary specifications and in a controlled-storage system for—controlled substances (in a locked warehouse).

In the second way, prefilling is meant as compounding, filling in a pharmaceutical compounding facility inside an already sterilized bag, preferably under vacuum or modified atmosphere conditions, wherein the test data in specific authorized labs) show stability duration of more than one month, time considered to be necessary for the patent's benefits in distribution, the avoidance of unnecessary moves by the nursing staff and the destruction of expired prefilled drugs owing to the snort stability duration. The present invention enables the normal operation of a pharmaceuticals warehouse. avoiding quick circulation (import-export) of drugs with short expiration times.

It is further preferred that the present invention pertains to a drug/medical device system, wherein the storage bag or the storage bag set is in the form of a drug cassette. The combination of the safe cassette and the pump is described in more detail in a copending patent application.

The present invention also involves an analgesic and/or an anaesthetic drug. An analgesic drug, or as briefly also named an analgesic is any member of the group of drugs used to achieve analgesia, e.g. relief from pain. Commonly known as painkillers, analgesic drugs act in various ways on the peripheral and central nervous systems. They are distinct from anesthetics, which reversibly eliminate sensation, Analgesic drugs include paracetamol (known in the US as acetaminophen or simply APAP), the non-steroidal anti-inflammatory drugs (NSAIDs) such as the salicylates, and opioid drugs such as morphine and opium. In choosing analgesics, the severity and response to other medication determines the choice of agent. Analgesic choice is particularly also determined by the type of pain, which also may influence the route and/or mode of administration.

It is preferred in the present invention that it pertains to a drug/medical device system, wherein the analgesic drug is selected from the group of the analgesic pharmaceutical substances such as morphine, oxycodone, fentanyl, sufentanil, pethidine, and/or the anaesthetic drug is selected from the group of the amino-amide group of anaesthetic pharmaceutical substances such as ropivacaine, lidocaine, bupivacaine, and/or from the ester-based group of anaesthetic pharmaceutical substances such as cloroprocaine.

It is further preferred in the present invention that it pertains to a drug/medical device system, wherein the system comprises a patient's feedback function and interface by which the analgesic effect is reported by the patient as a value within a range, or as an answer to more complex questions asked by the feedback function.

It is further preferred in the present invention that it pertains to a drug/medical device system, wherein the system comprises a control function for the patient's identification and the administration route verification before administration start up, and for providing a commensurate control report.

It is further preferred in the present invention that it pertains to a drug/medical device system, wherein the system comprises a safety function for monitoring the breathing rhythm with an indication and/or warning in case of wrong dosage administration.

It is additionally preferred in the present invention that it pertains to a drug/medical device system, wherein the storage bag comprises an one-dimensional or two-dimensional bar-code label/marking identification and/or RFID/NFC wireless identification of one or more data related to drug type, drug concentration, solvent, bag volume, drug batch, administration route, maximum limits for safe infusion per patient code, and optionally for wireless transmission of the infusion protocol. The term RFID has the meaning Radio Frequency Identifier. and the term NFC has the meaning Near Field Communication.

Radio-frequency identification (RFID) is the wireless non-contact use of radiofrequency electromagnetic fields to transfer data, for the purposes of automatically identifying and tracking tags attached to objects. Some tags require no battery and are powered and read at snort ranges via magnetic fields (electromagnetic induction). Others use a local power source and emit radio waves (electromagnetic radiation at radio frequencies). The tag contains electronically stored information which may be read from up to several meters away. Unlike a bar code, the tag does not need to be within line of sight of the reader and may be embedded in the tracked object.

Near field communication (NFC) is a set of standards for electronic devices, and for example, is also used for smartphones and similar devices, to establish radio communication with each other by touching them together or bringing them into close so proximity, usually no more than a few centimeters. Present and anticipated applications include contactless transactions, data exchange, and simplified setup of more complex communications such as WiFi. Communication is also possible between an NFC device and an unpowered NFC chip, called a “tag”. NFC standards cover communications protocols and data exchange formats, and are based on existing radio-frequency identification (RFID) standards including, for example, ISO/IEC 14443 and FeliCa. The NFC standards include, for example, ISO/IEC 18092 and those defined, promoted and certified for device compliance by the NFC Forum, which was founded in 2004, and now has more than 160 members.

It is further preferred in the present invention that it pertains to a drug/medical device system, wherein the system comprises a protection means against abuse and wherein the identification of the data content of the storage bag label/marking is carried out, preferably automatically, by a function of the pump, by a mobile application connected to or communicating with the pump locally, or by an internet application connected to or communicating with the pump locally, and wherein the integrated function of the pump, the mobile or internet application is enabled to unlock the storage bag, preferably the drug cassette, to be ready for use.

It is further preferred in the present invention that it pertains to a drug/medical device system, wherein system comprises a interface function through which the administration protocol, including but without limiting, the patient identification, the administration route, the drug label/marking content identification, the intermediate and/or final infusion protocol, the monitoring of analgesia level and/or other side-effect information, and the therapeutic results can be reported to a local network or the internet to the attending physician, and through which the attending physician may effect changes to the administration protocol within pre-set limits.

The RFID/NFC system found preferably on the bag label marking allows the rapid stock report per commodity, with massive reading, and the use of a long-range antenna for storing, as well as rapid counting and invoicing of batches for sale.

Furthermore, the RFID/NFC system mentioned below has preferably the ability to unlock a closed, hence safe, drug cassette only when it is placed on the pump and is authorized by the system for infusion. This way, safe drug cassettes may be stored at home, instead of the user procuring them, one by one, from the drugstore.

After prefilling and sterilization, a label or a marking is preferably placed on the backside with the same and/or additional information in order to avoid in-use errors, bearing a two-dimensional bar-code with hundreds of digital bytes stored in it, and/or chip with an RFID (Radio Frequency Identifier) or NFC (Near Field Communication) antenna operating without the use of a battery.

The information may include one or more of the following: drug type, concentration, type of solvent, bag volume, production lot in case an easy recall is required, highest flow and volume/safety limits for a specific patient code, preferred infusion protocol, administration route.

Reading labeldata may be executed through a RFID/NFC reader integrated into the infusion pump, or through a mobile application reading a bar-code via its photo camera feature, or NFC coming soon to at mobile phones enabling payments. A commercial mobile device, or one manufactured by our company, would be capable of transmitting label data via WiFi or GSM/GPRS. to the internet and the treatment server, and via WiFi to the pump itself, the pump also including WiFi of particularly low consumption.

Direct one to one information provision to the pump from the drug label/marking, concerning the drug type and concentration enables the right and/or automatic choice of the infusion protocol, and the avoidance of errors being too frequent at present.

Furthermore, through the mobile application WiFi interface and the treatment server, additional information is arriving to the pump, relating to patient's name, his safety limits code, his identification through a direct question to the pump (are you patient Mr. XXX?), or patient bar-code, or identification of the patient's biometric data by the mobile application, and the final infusion protocol, and the control of the administration route by the patient. Patients are classified based on their age; weight etc., as regards their resistance to analgesics, and this code is used by the pump to compute safety infusion limits for each separate drug according to the information on the label/marking.

It is obvious that in utilizing the above automation that avoids (reduces) errors and may be carried out from the patient's setting, and the collection of the prefilled bag from the neighbourhood pharmacy store, and its easy aseptic placement (i.e., no special skills or license required) on the pump (comprising one set plus drug), by implementing the present invention, the high-cost transfer of nursing staff at home is no longer necessary, thus enabling money saving.

In addition, in the present invention a special mattress/sheet may be used having specific sensors for measuring heart beat, respirations per minute and spasms (pain). This layer is connected to the rest of the system, preferably via WiFi, and controls any tardiness Or temporary loss of breath in order to sound an alarm or to lower the dose automatically, or through the intervention of a physician or both, Le automatically at first and then notifying the nursing staff for a definitive correction. In order to increase the dosage administered, the mattress feedback as well as questions addressed to the patient or his environment may be utilized.

1. Drug/medical device system for administering an analgesic and/or an anaesthetic drug to a patient preferably via the IV route or the central nerve block or the peripheral nerve block comprising
(a) a pump for administering at least one analgesic and/or anaesthetic drug,
(b) a storage bag or a storage bag set prefilled with the at least one analgesic and/or anaesthetic drug, and
(c) at least one means whereby the pump (a) and the storage bag or storage bag set (b) are designed to exclusively cooperate with each other when administered to an individual patient,
and wherein the method used to provide the pre-filled storage bag or a storage bag set (b) is in compliance with the guidelines of a competent drug regulatory authority applicable for pre-filled systems, preferably for pre-filled systems for long-term storage.

2. Drug/medical device system according to claim 1, wherein the storage bag is made of a multilayer film that has a—surface in contact with the drug made of a material selected from the group of ethylenevinylacetate (EVA), and preferably a linear polyolefinic polymer, and/or a cyclic polyolefinic polymer, and further inner layers made of barrier materials to moisture, gases and UV light.

3. Drug/medical device system according to claim 1, comprising a protecting storage case made of cardboard or expanded polystyrene or other foam sheets.

4. Drug/medical device system according to claim 1, wherein the storage bag comprises internal striations, preferably internal micro-striations or any other embossment or texture that makes the surface in contact with the drug not completely flat.

5. Drug/medical device system according to claim 1, wherein the storage bag is adjusted for portable use, and preferably wherein a single storage bag is equipped with an individual external packaging and/or a multitude of storage bags is equipped with a collective packaging.

6. Drug/medical device system according to claim 1, wherein the storage bag or the storage bag set is in the form of a drug cassette.

7. Drug/medical device system according to claim 1, wherein the analgesic drug is selected from the group of the analgesic pharmaceutical substances such as morphine, oxycodone, fentanyl, sufentanil, pethidine, and/or the anaesthetic drug is selected from the group of the amino-amide group of anaesthetic pharmaceutical substances such as ropivacaine, lidocaine, bupivacaine, and/or from the ester-based group of anaesthetic pharmaceutical substances such as cloroprocaine.

8. Drug/medical device system according to claim 1, wherein the system comprises a patient's feedback function and interface by which the analgesic effect is reported by the patient as a value within a range, or as an answer to more complex questions asked by the feedback function.

9. Drug/medical device system according to claim 1, wherein the system comprises a control function for the patient's identification and the administration route verification before administration start up, and for providing a commensurate control report.

10. Drug/medical device system according to claim 1, wherein the system comprises a safety function for monitoring the breathing rhythm with an indication and/or warning in case of wrong dosage administration.

11. Drug/medical device system according to claim 1, wherein the storage bag comprises a one-dimensional or two-dimensional bar-code label/marking identification and/or RFID/NFC wireless identification of one or more data related to drug type, drug concentration, solvent, bag volume, drug batch, administration route, maximum limits for safe infusion per patient code, and optionally for wireless transmission of the infusion protocol.

12. Drug/medical device system according to claim 11, wherein the system comprises a protection means against abuse and wherein the identification of the data content of the storage bag label/marking is carried out, preferably automatically, by a function of the pump, by a mobile application connected to or communicating with the pump locally, or by en internet application connected to or communicating with the pump locally, and wherein the integrated function of the pump, the mobile or interact application is enabled to unlock the storage bag, preferably the drug cassette, to be ready for use.

13. Drug/medical device system according to claim 12, wherein system comprises an interface function through which the administration protocol, including but without the patient identification, the administration route, the drug label content identification, the intermediate and/or final infusion protocol, the monitoring of analgesia level and/or other side-effect information, and the therapeutic results can be reported to a local network or the internet to the attending physician, and through which the attending physician may effect changes to the administration protocol within pre-set limits.
